(19) United States
(12) Patent Application Publication
(10) Pub. No.: US 2021/0187147 A1
(43) Pub. Date: Jun. 24, 2021

(54) Apparatus for Delivering a Scent

(71) Applicant: Charles F. O'Toole, III, Brookhaven, PA (US)

(72) Inventor: Charles F. O'Toole, III, Brookhaven, PA (US)

(21) Appl. No.: 17/247,565

(22) Filed: Dec. 16, 2020

Related U.S. Application Data
(60) Provisional application No. 62/949,937, filed on Dec. 18, 2019.

(51) Int. Cl. A61L 9/12; A62B 23/02

(52) U.S. Cl. CPC A61L 9/12; A62B 23/025

(57) ABSTRACT

An air-permeable body configured to hold a scenting means affixed to an article of clothing, to a personal protective mask or the like. The apparatus includes replaceable scenting means including packets or scented material dissolvable in air. A fastener, such as a clip, or magnet affixes the apparatus to fabric, an article of clothing or a personal protective clothing or a personal protective mask.

### TECHNICAL FIELD

The present disclosure relates to a scenting means configured to be attached to an article of clothing or a personal protective mask.

### BACKGROUND

The wearing of perfume and the enjoyment of various scents is common. Commonly, scents are used to create a pleasant environment. A specific use of scents is to cover unpleasant odors. One often comes in contact with unpleasant odors while wearing personal protective equipment.

Perfume intolerance or perfume allergy is a condition wherein people exhibit sensitivity or allergic reactions to ingredients in some perfumes and some other fragrances. It is considered a multiple chemical sensitivity.

A device for affixing an aerosol dissolvable scented material would overcome the aforementioned issues.

### SUMMARY

The present disclosure relates to an air-permeable body configured to hold a scenting means affixed to an article of clothing, to a personal protective mask or the like. The apparatus includes replaceable scenting means including packets or scented material dissolvable in air. A fastener, such as a clip, or magnet affixes the apparatus to fabric, an article of clothing or a personal protective clothing or a personal protective mask. The embodiment can be used in a number of areas in which a means for blocking out of unpleasant odors such as for hospital use, nursing homes, fire-fighting equipment, and mortuary and funeral home usage and many others areas. In another application of the apparatus, the device is affixed to an article of clothing to emit a scent as a pleasant fragrance or a scent to cover odors.

### BRIEF DESCRIPTION OF THE DRAWINGS

The references below are made to assist those of skill in the art in making and using the disclosed apparatus and method. The description is intended to define but not limit the scope of the invention.

FIG. 1 is an illustration of an example embodiment in a perspective, exploded view.

FIG. 2A is a perspective view showing the embodiment removably engaged with a piece of fabric.

FIG. 2B is a cross sectional view of the embodiment as illustrated in FIG. 2A.

FIG. 3 is a bottom, perspective view of an example embodiment used on a personal protective mask.

FIG. 4 is a rear, perspective view of an example embodiment used on a personal protective mask.

FIG. 5A is a detail view of an example embodiment worn on the cuff of a shirt.

FIG. 5B is a detail view of an example embodiment worn in a pocket of a shirt.

FIG. 6 is an illustration of an iteration of the embodiment in a perspective view

FIG. 7 is a perspective, exploded view of the embodiment of FIG. 5.

FIG. 8 is a rear, perspective view of the embodiment of FIG. 5, used on a personal protective mask.

FIG. 9A is a detail view of an example embodiment worn on the cuff of a shirt.

FIG. 9B is a detail view of an example embodiment worn in a pocket of a shirt.

### DESCRIPTION

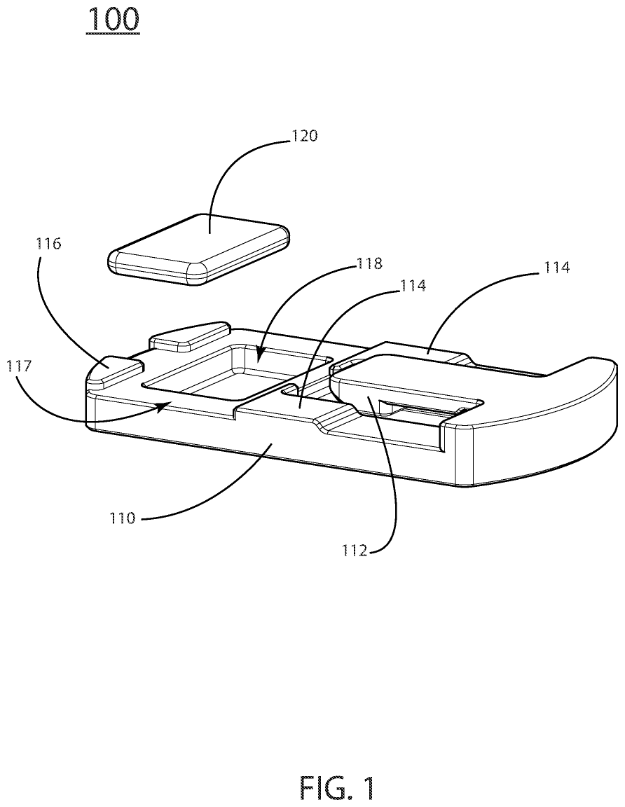

Referring to FIG. 1, in example embodiment 100; a body 110 is formed with a clip 112 and is contiguous with a receptacle 118 for holding a scent packet 120. Protrusions 116 and 114 provide a space 117 that allows air to flow past the receptacle 118 and scent pack 120. One skilled in the art understands that if the clip 112 were fastened to a piece of fabric such as an article of clothing or a personal protective mask, that an airspace 117 would prevent fabric from covering the exposed surface(s) of the scent pack 120 and would improve airflow, thus allowing scent to be dissolved in passing air.

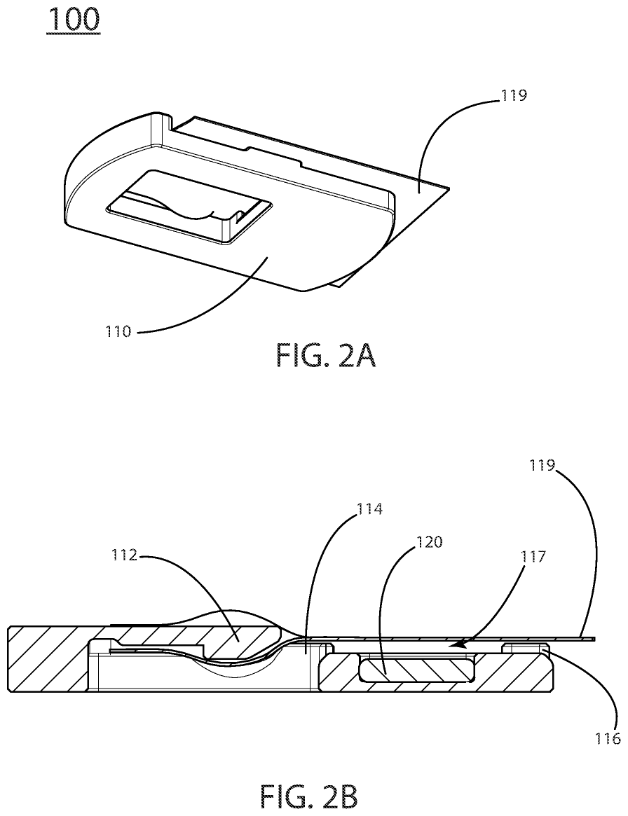

FIG. 2A is a bottom perspective view of the embodiment 100 clipped to a piece of fabric 119. FIG. 2B is a cross section view of the embodiment 100. The fabric 119 is held under the clip 1112 and rests on the protrusions 116 and 114, leaving space 117 for air to flow past the scent packet 120.

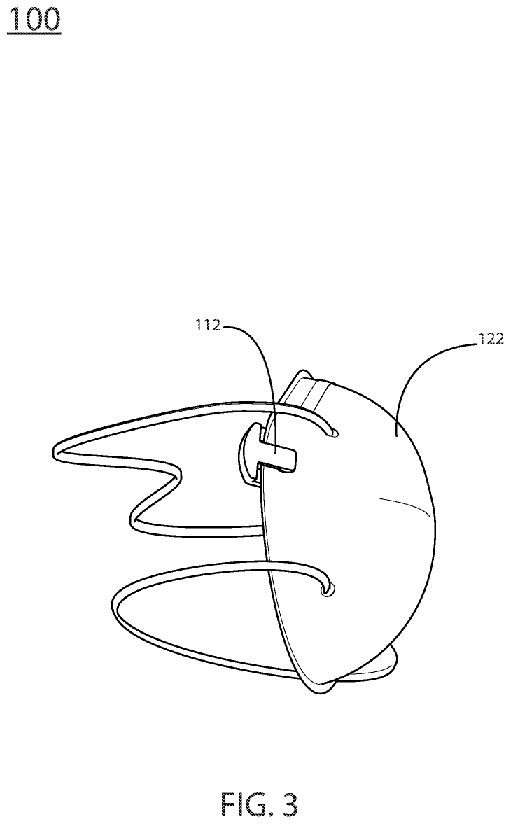

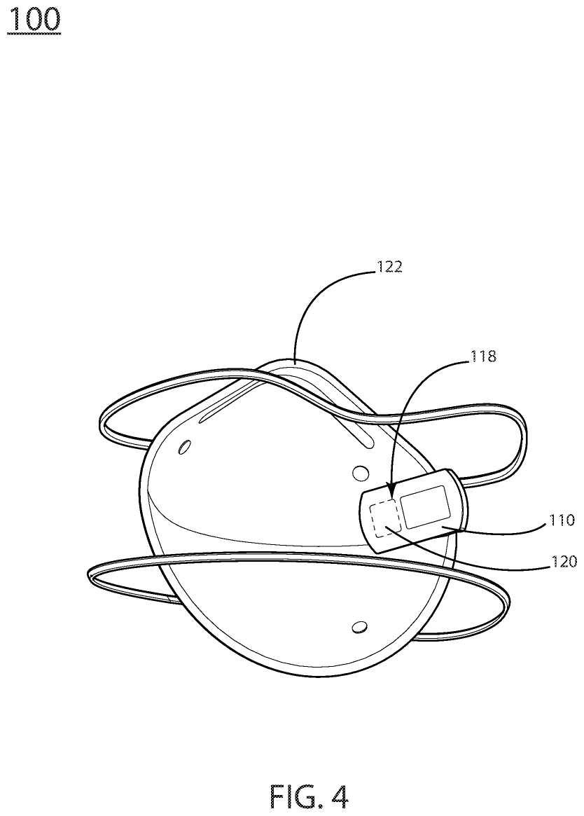

FIG. 3 and FIG. 4 are perspective views of the example embodiment 100 worn on a personal protective mask 122. The clip 112 resides on the outside of the mask and the body 110 and receptacle 118 reside inside the mask 122, where the scent packet is held inside the mask where a user may smell the scent emanating from the packet 120

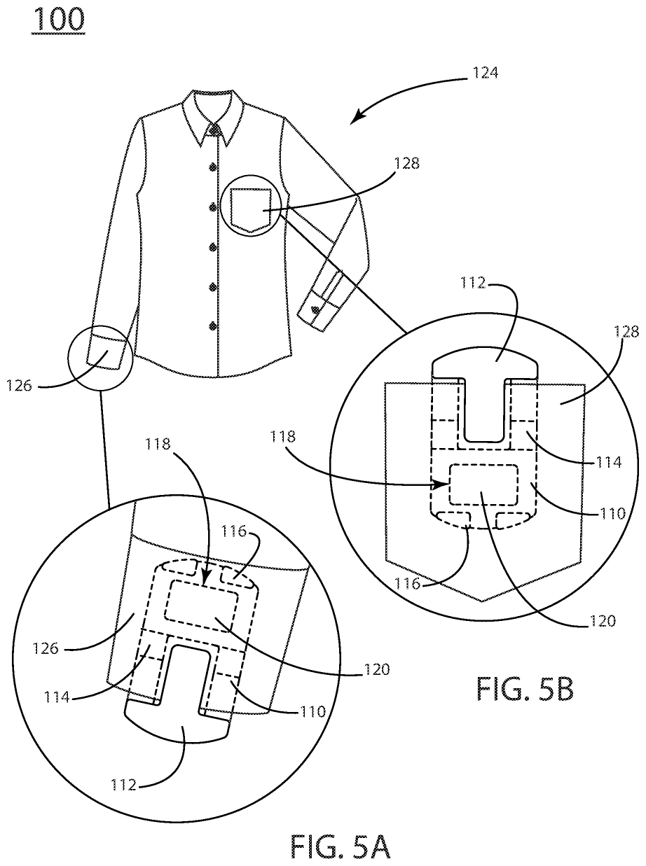

FIG. 5A depicts an example embodiment 100 worn on a shirt cuff 126, while FIG. 5B depicts the same example embodiment 100 worn in a shirt pocket 128. The clip 112 resides outside of the cuff 126 or pocket 128 while the body 110 resides inside the cuff 126 or pocket 128, thus supporting the receptacle 118 for the scent packet 120. One skilled in the art understands how protrusions 114 and 116 may keep fabric from covering a scent pack 120.

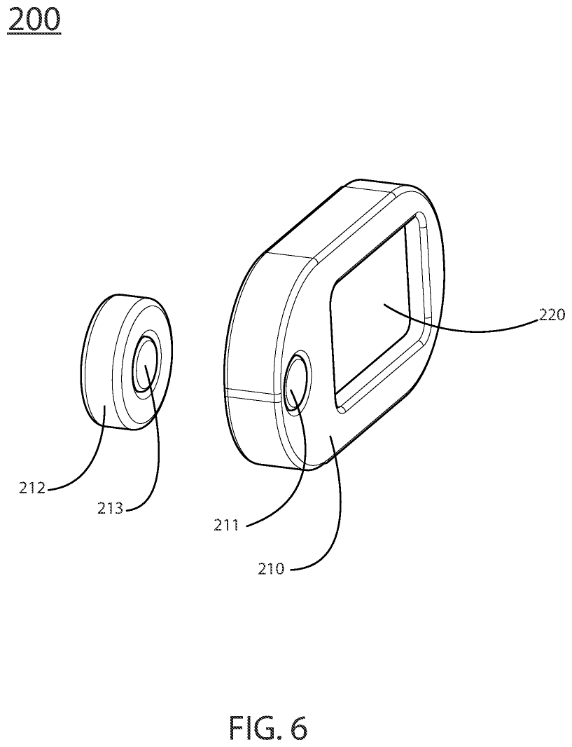

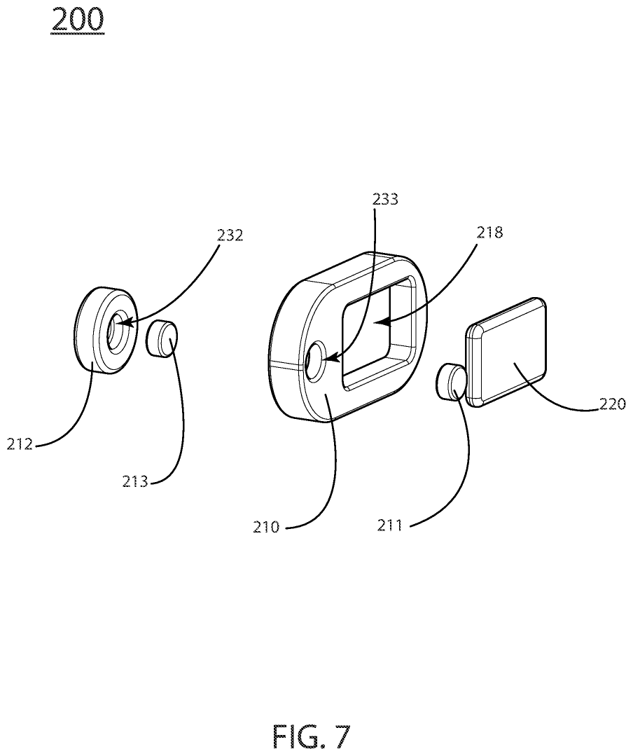

FIG. 6 is a partially exploded view of an iteration of the embodiment 200. FIG. 7 is an exploded view of the embodiment 200. A body 210 has a receptacle 218 for holding a scent packet 220. The body further has a receptacle 233 for holding a magnet 211. A magnet housing 212 has a receptacle 232 for a mating magnet 213. One skilled in the art understands that the mating magnet 213 and magnet housing 212 may be placed on the opposite side of a piece of fabric than the body 210 and magnet 211 thus attaching the apparatus to the piece of fabric.

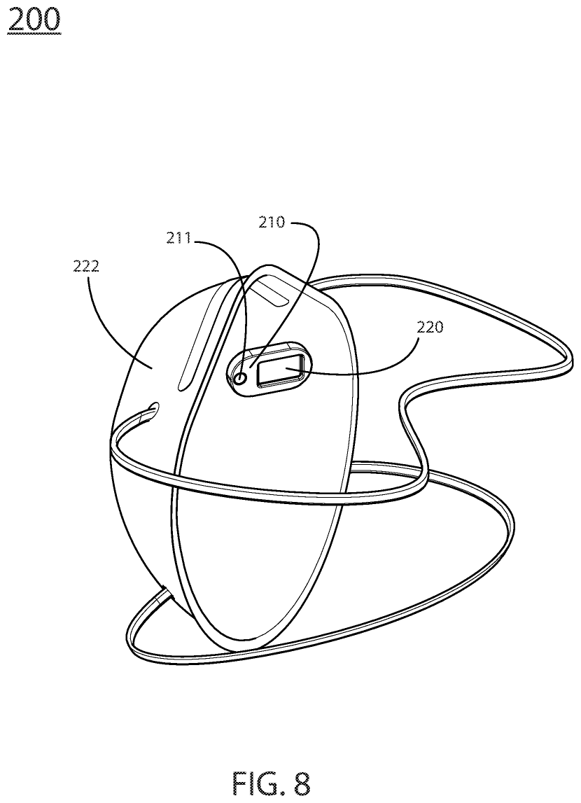

FIG. 8 is a rear perspective view showing the iteration of the embodiment 200 affixed to a personal protective mask 222. The body 210 holds the scent pack 220 against the inner surface of the personal protective mask 222 by the magnet 211, residing on the inside of the personal protective mask 222, that mates with the mating magnet 213 in the magnet housing 212 (FIG. 7) that resides on the outside of the personal protective mask.

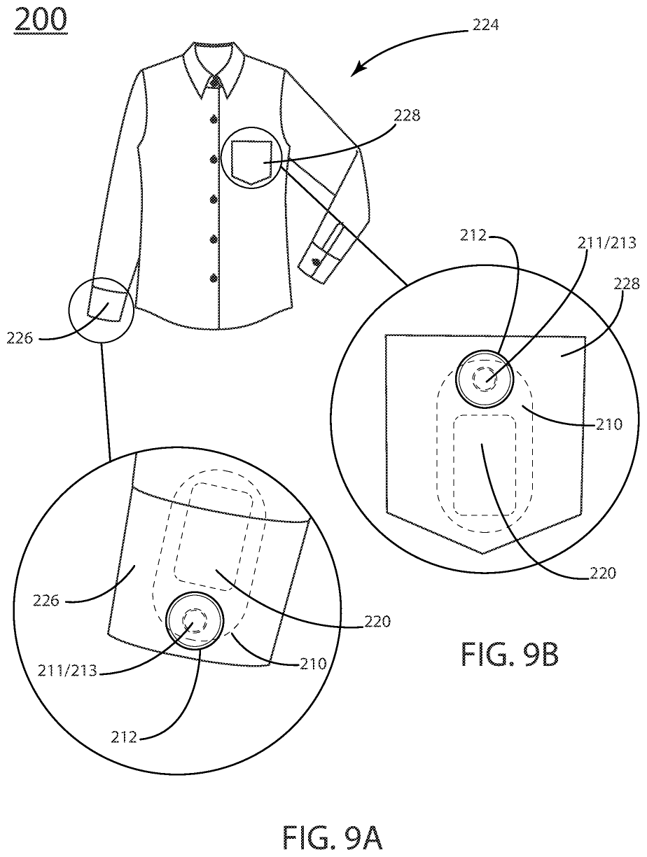

FIG. 9A depicts an example embodiment 200 worn on a shirt cuff 226, while FIG. 9B depicts the same example embodiment worn in a shirt pocket 228. The body 210 resides inside the cuff 226 or pocket 228 while the magnet housing and magnet 211/213 resides outside the cuff 226 or pocket 228, thus supporting the receptacle 218 for the scent packet 220. One skilled in the art understands how the scent packet may emit scent from the user without the user needing to apply perfume or other scented products directly to their skin.

1. An apparatus for delivering a scent comprising:
a body having a top and bottom, a left end and a right end; and
said body symmetrical about a central plane extending from said left end to said right end; and
a clip formed into said right end; and
said clip configured to removably engaged fabric; and
a receptacle formed into said left end; and
said receptacle configured to removably engage a packet filled with scented material dissolvable in air; wherein
the apparatus may be clipped to a piece of fabric on a person for delivering the scent about the ambient area.

2. The apparatus of claim 1 wherein the piece of fabric is a personal protective mask;
the apparatus worn on the inside of the mask proximal to the wearer's nose and mouth.

3. The apparatus of claim 1 wherein the piece of fabric is a portion of a garment; and the scented material is comprised of perfume.

4. The apparatus of claim 1 further comprising:
at least one protrusion proximal to said receptacle for preventing material from covering said packet filled with scented material; wherein
said at least one protrusion provides space for air to pass over said packet filled with scented material.

5. An apparatus for delivering a scent comprising:
a body having a top and bottom, a left end and a right end; and
said body symmetrical about a central plane extending from said left end to said right end; and
a first magnet fixedly engaged proximal to said right end; and
a receptacle formed into said left end; and
said receptacle configured to removably engage a packet filled with scented material dissolvable in air; and
a housing configured to hold a second magnet; and
said second magnet configured to reside on the opposite side of a piece of fabric and removably engaged with said first magnet thus removably engaging the apparatus with said piece of fabric; wherein
the apparatus may be removably engaged with a piece of fabric on a person for delivering the scent about the ambient area surrounding the person.

6. The apparatus of claim 1 wherein the piece of fabric is a personal protective mask; the apparatus worn on the inside of the mask proximal to the wearer's nose and mouth.
